(19) United States
(12) Patent Application Publication
(10) Pub. No.: US 2025/0082694 A1
(43) Pub. Date: Mar. 13, 2025

(54) PROBIOTICS FOR USE IN BOOSTING THE IMMUNE SYSTEM

(71) Applicant: INTERNATIONAL N&H DENMARK APS, Kongens Lyngby (DK)

(72) Inventors: MARKUS LEHTINEN, KANTVIK (FI); SINIKKA ANNELI LATVALA, KANTVIK (FI); LIISA LEHTORANTA, KANTVIK (FI)

(21) Appl. No.: 18/580,713

(22) PCT Filed: Jul. 13, 2022
(86) PCT No.: PCT/EP2022/069586

§ 371 Date: Jan. 19, 2024

(30) Foreign Application Priority Data
Jul. 22, 2021 (EP) 21187190.0

(51) Int. Cl. A61K 35/741; A61P 37/04

(52) U.S. Cl. CPC A61K 35/741; A61P 37/04

(57) ABSTRACT

This invention relates to bacterial strains, compositions comprising bacterial strains as well as methods and uses of said strains and compositions for use in boosting the immune system. More specifically, the invention relates to bacterial strains having immunomodulatory properties providing specific health benefits in a subject in need thereof.

### FIELD OF THE INVENTION

This invention relates to bacterial strains for use in boosting the immune system, and more specifically, the invention relates to bacterial strains having immunomodulatory properties providing specific health benefits. The invention also relates to the specific use of probiotics in boosting and modulating anti-viral responses.

### BACKGROUND OF THE INVENTION

In the past decade, the association of the microbiota in regulating human immune responses and homeostasis has been established. Furthermore, immune homeostasis and inflammatory tone of the host is associated with several diseases and with health benefits, suggesting that influencing microbiota and the immune system can influence human health. The effects of the microbiota on the immune system are largely unknown, however, diversity of the microbiota is one of the key indicators of healthy microbiota.

Probiotics are defined as live microorganisms that when administered in adequate amounts confer a health benefit to the host (Hill 2014). Probiotics offer means to influence the microbiota ecosystem and thus, indirectly, the immune function. Probiotics also offer means to directly influence the immune system by the uptake of the probiotics by the intestinal lamina propria through epithelium or via specialized M cells. Similar structures and microbiome-host immune system interactions are also found at other mucosal sites such as mouth and respiratory tract, as well as in reproductive organs.

Mucosal immune system recognizes micro-organisms and react to them by secreting cytokines, such as anti-inflammatory interleukin (IL)-10, and pro-inflammatory or immunomodulatory interferon gamma (IFN-γ), interleukin IL-1β, IL-6, IL-12 (also called IL-12p70), IL-23, transforming growth factor (TGF)-β, and tumor necrosis factor (TNF)-α. The secretion and balance of the cytokine production is important in fighting infectious diseases, but also in managing the development of allergic diseases and regulating the inflammatory tone.

Macrophages are key cells for intestinal and systemic inflammatory status. They recognize the surrounding cytokine and microbial metabolite environment that drives their inflammatory response. Upon recognizing a bacterium, e.g. probiotic or pathogen, macrophages respond accordingly with either more pro- or anti-inflammatory manner. Thus, high load of potentially harmful bacteria or dysbiosis increases cytokine production and drives inflammation. On the other hand, probiotics and commensals may help alleviate the inflammatory status by making the macrophages respond in a more anti-inflammatory fashion. Markers of macrophage activation are cytokines IL-12 and IFN-g whereas IL-10 suppresses the inflammatory response.

The function of the dendritic cells (DC) is to modulate adaptive immune responses to different micro-organisms. DC's phagocytose and recognize the micro-organisms and change their cytokine and gene expression accordingly. DC's then instruct T helper (Th) cells to differentiate into Th1, Th2, Th17 or regulatory T cells (Treg). Each Th type is more equipped to respond to different threats: Th1 to cancer, viruses and intracellular bacteria, Th2 to parasites, Th17 to extracellular bacteria and fungi, while Treg responses are regulatory or homeostatic in nature. DC's, once meeting a micro-organism, are polarized to drive certain T cell responses. Markers of DC polarization to different Th programs are (1) IL-12 and IFNg for Th1, (2) IL-4 for Th2, (3) IL-6 and IL-23 for Th17, and (4) IL-10 for regulatory responses.

Modulation of immune function by probiotics could thus provide benefits to humans. However, the strains have specific effects and are more beneficial towards certain conditions, depending on their impact on immune cells that can be measured as cytokine profiles.

The health benefits can be on infectious diseases that are caused by different types of infective micro-organisms that threaten the host—bacteria, viruses, parasites and fungi. Against each type of the threat certain immune response type is generated as was discussed above.

One type of infectious disease are viral respiratory infections that typically caused by rhinoviruses, coronaviruses, influenza, and adenoviruses. These may be collectively called as common cold.

Viruses typically induce immune responses by engaging toll like receptors (TLR), such as TLR3, TLR7 and TLR8. TLR's can be also artificially induced in vitro with polyinosinic:polycytidylic acid (polyI:C) and resiquimod (R848) that mimics anti-viral responses in the human cells.

Severe viral respiratory infections can lead to an overactivation of inflammatory responses. The cytokine storm can be fatal and has been speculated to be driven by macrophages. Respiratory viruses may cause this condition but is typically observed with coronaviruses such as Severe Acute Respiratory Syndrome (SARS) CoV, and SARS-COV2, MERS, and influenza.

Inflammatory diseases are caused by an uncontrolled inflammatory process that can be due to immune system dysfunction or regulation, leaky gut, cellular senescence, chronic infection, or some other metabolic or physiological problem. It can basically influence any human organ. The condition is typically characterized by slow increase in inflammatory mediator levels. Some common conditions where low grade inflammation can be found are: obesity, inflammatory bowel syndrome, diabetes, fatty liver disease, rheumatoid arthritis, osteoarthritis, asthma, endometriosis, Alzhemeimer's diseases and Parkinson's disease.

Allergic diseases are a group of diseases where immune system is hypersensitive to harmless environmental antigens such as pollen. Allergic diseases include food allergies, asthma, atopic dermatitis, and allergic rhinitis. Immunologically rise of the allergic diseases are characterize by overactive Th2 responses and cytokine secretion. Mechanistically, Th1 type cytokines IL-12 and IFNg; and Treg cytokine IL-10 can suppress Th2 cytokine secretion.

### OBJECT OF THE INVENTION

In order to overcome the current challenges presented by infectious diseases, the inventors have shown that the probiotic bacteria as described in the present invention can be used in boosting the immune system in a subject in need thereof.

It is therefore an object of the present invention to provide probiotic bacteria as described in the present invention, a method, as well as compositions comprising such bacterial strains, to be used in boosting the immune system in a subject in need thereof.

It is another object of the present invention to provide bacterial strains, a method, as well as compositions comprising such bacterial strains, to be used in preventing, treating and/or reducing infections and symptoms associated with infectious diseases in a subject in need thereof.

It is another object of the present invention to provide bacterial strains, a method, as well as compositions comprising such bacterial strains, to be used in preventing, treating and/or reducing inflammation and symptoms associated with inflammatory diseases in a subject in need thereof.

It is another object of the present invention to provide bacterial strains, a method, as well as compositions comprising such bacterial strains, to be used in preventing, treating and/or reducing inflammation and symptoms associated with allergic diseases in a subject in need thereof.

It is a further object of the present invention to provide bacterial strains, a method, as well as compositions comprising such bacterial strains, to be used in preventing, treating and/or reducing viral infections and symptoms associated with viral infectious diseases in a subject in need thereof.

### SUMMARY OF THE INVENTION

In one aspect, the invention relates to a probiotic composition comprising one or more bacterial strain selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri, for use in boosting the immune system in a subject.

In another aspect, the present invention relates to a method of boosting the immune system in a subject, said method comprising administering a probiotic composition comprising one or more bacterial strain selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri to said subject.

In yet a further aspect, the present invention relates to a use of a probiotic composition comprising one or more bacterial strain selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri, for boosting the immune system in a subject.

### DETAILED DISCLOSURE OF THE INVENTION

The detailed aspects of this invention are set out below. Some of the detailed aspects are discussed in separate sections. This is for ease of reference and is in no way limiting. All of the embodiments described below are equally applicable to all aspects of the present invention unless the context specifically dictates otherwise.

### Bacteria

The bacterial strains of the present invention are selected from bacterial strains of the genera Lactiplantibacillus, Lacticaseibacillus, Streptococcus, Lactobacillus, Levilactobacillus, Lactococcus, Bifidobacterium, Ligilactobacillus, Limosilactibacillus.

Preferably, the bacterial strains of the present invention are selected from bacterial strains of the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri.

In particular, the bacterial strains of the present invention are chosen from the strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

The strains can be used individually or in combination and, for example, in food products, food ingredients, dietary supplements, vaccines or in pharmaceutical acceptable compositions or formulations.

Preferably, the bacterial strains used in the present invention are bacterial strains which are generally recognised as safe and which are preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

In one embodiment, the present invention relates to a probiotic composition comprising one or more bacterial strain selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri, for use in boosting the immune system in a subject.

The term “subject”, as used herein, means a mammal, including for example livestock (for example cattle, horses, pigs, and sheep) and humans. In one embodiment the subject is a human. In one embodiment the subject is female. In one embodiment the subject is male. In another embodiment, the subject is a dog (such as a member of the genus Canis) or a cat (such as a member of the genera Felis or Panthera).

In a particular embodiment, the immune system is innate immune system.

In another embodiment, the innate immune system affects viral and/or bacterial action and/or cancer cells.

In particular, the innate immune system controls the pro-inflammatory response and/or low-grade inflammation. More particularly, the immune system effect on the viral and/or bacterial action and/or on cancer cells and the innate immune system control of the pro-inflammatory response and/or low-grade inflammation is due to activation of macrophages.

In another embodiment, the boosting of the immune system modulates the immune function. The modulation of the immune system boosts anti-pathogen and/or anti-cancer immunity.

In particular, the modulation of the immune system boosts immunity against extracellular bacteria and fungi. The modulation of the immune system also bosts regulatory responses. Said boosting is due to activation of dendritic cells.

In another embodiment of the present invention, the dendritic cells activate T-cells. In particular, the T-cells are T-helper cell type 1 (Th1) and/or T-helper cell type 17 (Th17) And/or regulatory T-cells (Treg).

In a further embodiment, the boosting of the immune system according to the present invention modulates anti-viral immunity. In particular, the modulation of the anti-viral immunity affects anti-viral response. More particularly, the modulation of the anti-viral immunity inhibits/controls the inflammation during the anti-viral response.

In a further embodiment, the modulation is due to the activation of macrophages.

According to one embodiment of the present invention, the modulation of the anti-viral immunity inhibits macrophage activation.

In a further embodiment, the modulation is due to the activation of dendritic cells. The dendritic cells activate the T-helper cell type 1 (Th1). The dendritic cells also activate the T-helper cell type 17 (Th17).

In another embodiment according to the present invention, the boosting of the immune system results in antigen specific immune responses. In particular, the boosting of the immune system is due to activation of antigen presenting cells.

In a further embodiment, the boosting of the immune system according to the present invention is due to activation of dendritic cells.

In a further embodiment, the boosting of the immune system is due to activation of macrophages.

The antigen presenting cells and/or the dendritic cells and/or the macrophages induce activation of antigen specific T cells. The antigen specific T cells are T-helper cell type 1 (Th1) and/or T-helper cell type 17 (Th17) and/or regulatory T-cells (Treg).

In one embodiment, the antigen is derived from the genome of said probiotic. In another embodiment, the antigen is not derived from the genome of said probiotic and is transferred to a probiotic strain.

In a particular embodiment of the present invention, the bacterial strains of the species Lactiplantibacillus plantarum are strains Lp-115, Lp12418 and/or Lp12407; the bacterial strains of the species Lacticaseibacillus paracasei are strains Lpc-37 and/or Lc-10; the bacterial strain of the species Lacticaseibacillus rhamnosus is strain Lr-32; the bacterial strain of the species Lacticaseibacillus casei is strain Lc-11; the bacterial strain of the species Streptococcus thermophilus is strain St-21; the bacterial strain of the species Lactobacillus gasseri is strain Lg-36; the bacterial strains of the species Lactobacillus acidophilus is strain La-14; the bacterial strain of the species Lactobacillus crispatus is strain Lx1220; the bacterial strain of the species Lactobacillus bulgaricus is strain Lb-87; the bacterial strain of the species Levilactobacillus brevis is strain Lbr-35; the bacterial strain of the species Lactococcus lactis is strain Ll-23; the bacterial strain of the species Bifidobacterium longum subsp. infantis is strain Bi-26; the bacterial strains of the species Bifidobacterium animalis subsp. lactis are strain B420, and Bi-07; the bacterial strain of the species Bifidobacterium longum is strain Bl-05; the bacterial strain of the species Bifidobacterium bifidum is strain Bb-06; the bacterial strain of the species Ligilactobacillus salivarius is strain Ls-33; the bacterial strain of the species Limosilactibacillus fermentum is strain SBS-1 and the bacterial strain of the species Limosilactibacillus reuteri is strain 1E1.

The bacterial strains of the present invention are all commercially available from DuPont Nutrition Biosciences ApS.

The bacterial strains were also deposited by DuPont Nutrition Biosciences ApS, of Langebrogade 1, DK-1411 Copenhagen K, Denmark, in accordance with the Budapest Treaty at the Leibniz-Institut Deutsche Sammlung von Mikroorganismen und Zellkulturen GmbH (DSMZ), Inhoffenstrasse 7B, 38124 Braunschweig, Germany, where they are recorded under the following registration numbers:
1. Strain Lp-115 (DGCC4715); deposited on 9 Feb. 2009 under registration number DSM22266.
2. Strain Lp-12418 (DGCC12418); deposited on 27 Sep. 2017 under registration number DSM32655.
3. Strain Lp-12407 (DGCC12407); deposited on 27 Sep. 2017 under registration number DSM32654.
4. Strain Lpc-37 (DGCC4981); deposited on 5 Oct. 2017 under registration number DSM32661.
5. Strain Lc-10 (DGCC4415); deposited on 1 Jun. 2021 under registration number DSM33881.
6. Strain Lr-32 (DGCC9913); deposited on 15 Jan. 2009 under registration number DSM22193.
7. Strain Lc-11 (DGCC9864); deposited on 1 Jun. 2021 under registration number DSM33885.
8. Strain St-21 (DGCC7693); deposited on 23 Feb. 2021 under registration number DSM33829.
9. Strain Lg-36 (DGCC10687); deposited on 1 Jun. 2021 under registration number DSM33879.
10. Strain La-14 (DGCC11491); deposited on 1 Jun. 2021 under registration number DSM33880.
11. Strain Lx1220 (DGCC4299); deposited on 29 Jul. 2015 under registration number DSM32100.
12. Strain Lb-87 (DGCC11687); deposited on 1 Jun. 2021 under registration number DSM33884.
13. Strain Lbr-35 (DGCC9912); deposited on 1 Jun. 2021 under registration number DSM33882.
14. Strain Ll-23 (DGCC8656); deposited on 23 Feb. 2021 under registration number DSM33830.
15. Strain Bi-26 (DGCC11473); deposited on 23 Feb. 2021 under registration number DSM33832.
16. Strain B420 (DGCC420); deposited on 30 Jun. 2015 under registration number DSM32073.
17. Strain Bi-07 (DGCC12895); deposited on 19 May 2020 under registration number DSM33526.
18. Strain Bl-05 (DGCC9917); deposited on 1 Jun. 2021 under registration number DSM22092.
19. Strain Bb-06 (DGCC10692); deposited on 1 Jun. 2021 under registration number DSM33896.
20. Strain Ls-33 (DGCC9868); deposited on 23 Feb. 2021 under registration number DSM33831.
21. Strain SBS-1 (DGCC1925); deposited on 29 Jul. 2015 under registration number DSM32105.
22. Strain 1E1 (DGCC10474); deposited on March 2015 under registration number ATCC: PTA-6509.

In one embodiment, the probiotic composition according to the present invention comprises at least 2 bacterial strains. In another embodiment, the composition comprises at least 3 bacterial strains. In a further embodiment, the composition comprises at least 4 bacterial strains. In another embodiment, the composition comprises at least 5 bacterial strains.

In a further embodiment, the probiotic composition of the present invention is a food product, a food ingredient, a dietary supplement, a vaccine or a pharmaceutical acceptable composition.

The invention further provides a mutant, a variant and/or a progeny of the bacterial strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

As used herein, the term “mutant” refers to any microorganism resulting from modification of the strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1. For example, a mutant may be a microorganism resulting from genetically modifying these strains.

As used herein, the term “variant” refers to a naturally occurring microorganism which is derived from the strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1. For example, a variant may be a microorganism resulting from adaption to a particular environment or cell culture conditions.

As used herein, the term “progeny” means any microorganism resulting from the reproduction or multiplication of any one of the strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1. Therefore, “progeny” means any direct descendant of any one of these strains. As such, the progeny strain may itself be identified as the same strain as the parent strain (i.e. strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1). It will be apparent to one skilled in the art that due to the process of asexual reproduction, a progeny strain will be genetically virtually identical to the parent strain. Accordingly, in one embodiment, the progeny may be genetically identical to the parent strain and may be considered to be a “clone” of the parent strain. Alternatively, the progeny may be substantially genetically identical to the parent strain.

The mutant, variant or progeny may have at least 90, 95, 98, 99, 99.5 or 99.9% sequence identity over the entire length of the bacterial genome with their parent strain. Furthermore, the mutant, variant or progeny will retain the same phenotype as the parent strain, for example the mutant, variant or progeny may demonstrate the same or equivalent effect on boosting the immune system in a subject as described in the present invention.

### Compositions

While it is possible to administer bacterial strains of the genera Lactiplantibacillus, Lacticaseibacillus, Streptococcus, Lactobacillus, Levilactobacillus, Lactococcus, Bifidobacterium, Ligilactobacillus and Limosilactibacillus alone according to the present invention (i.e., without any support, diluent or excipient), the bacterial strains of Lactiplantibacillus, Lacticaseibacillus, Streptococcus, Lactobacillus, Levilactobacillus, Lactococcus, Bifidobacterium, Ligilactobacillus, Limosilactibacillus are typically administered on or in a support as part of a product, in particular as a component or at least as one of the components of a food product, a food ingredient, a dietary supplement, a vaccine or a pharmaceutical acceptable composition. These products typically contain additional components well known to those skilled in the art.

In one embodiment, the present invention relates to a composition comprising one or more bacterial strains chosen from the genera Lactiplantibacillus, Lacticaseibacillus, Streptococcus, Lactobacillus, Levilactobacillus, Lactococcus, Bifidobacterium, Ligilactobacillus and Limosilactibacillus. In a particular embodiment, the composition comprises one or more bacterial strain chosen from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri.

In a particular embodiment, the present invention relates to a composition comprising one or more of the bacterial strains chosen from strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

In a particular embodiment, the present invention relates to a composition comprising at least 2 bacterial strains chosen from strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

In a particular embodiment, the present invention relates to a composition comprising at least 3 bacterial strains chosen from strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

In a particular embodiment, the present invention relates to a composition comprising at least 4 bacterial strains chosen from strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

In a particular embodiment, the present invention relates to a composition comprising at least 5 bacterial strains chosen from strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

The subject being treated with the composition has or can have one or more pre-existing conditions selected from the group consisting of obesity, type II diabetes, chronic lung disease or moderate to severe asthma, heart conditions, immunocomprimised, chronic kidney disease and liver disease.

In one embodiment, the subject is 65 years of age or older and/or is a resident in a nursing home or long-term care facility.

The composition as described in the present invention, when used and administered to the subject boosts the immune system in said subject.

In another embodiment, the present invention relates to a method of boosting the immune system in a subject in need thereof, said method comprising administering a probiotic composition according to the present invention comprising one or more bacterial strain selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalissubsp. lactis, Bifidobacterium longum, Bifidobacterium bifidum, Ligilactobacillus salivarius, Limosilactibacillus fermentum and Limosilactibacillus reuteri.

In a further embodiment, the present invention relates to a method of boosting the immune system in a subject in need thereof, said method comprising administering a probiotic composition according to the present invention comprising one or more bacterial strain selected from the strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1.

### Dosage

The bacterial strains used in accordance with the present invention may be present from 10⁶ to 10¹⁴ CFU of bacteria/g of support, and more particularly from 10⁸ to 10¹² CFU of bacteria/g of support, preferably 10⁹ to 10¹² CFU/g of support.

Suitably, the bacterial strains used in accordance with the present invention, such as strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1, may be administered at a dosage of from about 10⁶ to about 10¹⁴ CFU of microorganism/dose, preferably about 10⁸ to about 10¹² CFU of microorganism/dose and more preferably from about 10⁹ to about 10¹¹ CFU of microorganism/dose. By the term “per dose” it is meant that this amount of microorganism is provided to a subject either per day or per intake, preferably per day. For example, if the microorganisms are to be administered in a food product, for example in a yoghurt, then the yoghurt will preferably contain from about 10⁸ to 10¹² CFU of the microorganism. Alternatively, however, this amount of microorganism may be split into 5 multiple administrations each consisting of a smaller amount of microbial loading-so long as the overall amount of microorganism received by the subject in any specific time, for instance each 24-hour period, is from about 10⁶ to about 10¹² CFU of microorganism, preferably 10⁸ to about 10¹² CFU of microorganism and more preferably from about 10⁹ to about 10¹¹ CFU of microorganism.

In accordance with the present invention an effective amount of at least one strain of a microorganism may be at least 10⁶ CFU of microorganism/dose, preferably from about 10⁶ to about 10¹² CFU of microorganism/dose, preferably about 10⁸ to about 10¹² CFU of microorganism/dose.

In one embodiment, preferably the bacterial strains, such as strains Lp-115, Lp12418, Lp12407, Lpc-37, Lc-10, Lr-32, Lc-11, St-21, Lg-36, La-14, Lx1220, Lb-87, Lbr-35, Ll-23, Bi-26, B420, Bi-07, Bl-05, Bb-06, Ls-33, SBS-1 and 1E1, may be administered at a dosage of from about 10⁶ to about 10¹⁴ CFU of microorganism/day, preferably about 10⁸ to about 10¹² CFU of microorganism/day, more preferably about 10⁹ to about 10¹¹ CFU of microorganism/day. Hence, the effective amount in this embodiment may be from about 10⁶ to about 10¹⁴ CFU of microorganism/day, preferably about 10⁸ to about 10¹² CFU of microorganism/day, more preferably about 10⁹ to about 10¹¹ CFU of microorganism/day.

By “support” is meant a composition, a food product, a food ingredient, a dietary supplement or a pharmaceutically acceptable composition. CFU stands for “colony-forming units”.

### Food Product

In one embodiment, the bacterial strains are used according to the invention in a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

When used as, or in the preparation of, a food, such as functional food, the bacteria of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the bacteria of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, vegetable milk, acidified soy/juice beverage, aseptic/retorted chocolate drink, bar mixes, beverage powders, calcium fortified soy/plain and chocolate milk, calcium fortified coffee beverage

Advantageously, where the product is a food product, the bacterial strains should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment age.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Dietary Supplements

The compositions of the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help prevent or treat diseases.

### Medical Food

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Composition

The bacteria of the present invention may be used as—or in the preparation of—a pharmaceutical composition or formulation. Here, the term “pharmaceutical” is used in a broad sense—and covers pharmaceuticals for humans as well as pharmaceuticals for animals (i.e. veterinary applications).

In a preferred embodiment, the pharmaceutical acceptable composition is a medicament.

The pharmaceutical composition can be for therapeutic purposes-which may be curative or palliative or preventative in nature. The pharmaceutical composition may even be for diagnostic purposes.

In a preferred embodiment of the present invention, the medicament is for oral administration.

A pharmaceutically acceptable composition or support may be for example a formulation or support in the form of creams, foams, gels, lotions, and ointments of compressed tablets, tablets, capsules, ointments, suppositories or drinkable solutions.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

Yeasts in general have been shown to be adjuvants in oral administration. Yarrowia, in particular, has been shown to drive the correct IL-12/Th1/interferon gamma path as well as inducing IL-27 leading to CD8 Cytotoxoc T-Lymphocyte synthesis/activation. It also appears to induce IL-17 production by the Th17 cell subset, normally involved in innate immunity of the gut epithelium including wall integrity.

Therefore, in a particular embodiment, the adjuvant is a yeast and, more particularly, yarrowia.

The pharmaceutical may be in the form of a solution or as a solid—depending on the use and/or the mode of application and/or the mode of administration.

The bacterial strains of the present invention may be used as pharmaceutical ingredients. Here, the composition may be the sole active component, or it may be at least one of a number (i.e. 2 or more) of active components.

The pharmaceutical ingredient may be in the form of a solution or as a solid—depending on the use and/or the mode of application and/or the mode of administration.

The bacterial strains may be used according to the present invention in any suitable form—whether when alone or when present in a combination with other components or ingredients. Likewise, combinations comprising the bacteria of the present invention and other components and/or ingredients (i.e. ingredients—such as food ingredients, functional food ingredients or pharmaceutical ingredients) may be used in any suitable form.

The bacterial strains may be used according to the present invention in the form of solid or liquid preparations or alternatives thereof. Examples of solid preparations include, but are not limited to tablets, capsules, dusts, granules and powders which may be wettable, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the bacteria of the present invention are used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid 30 monoglycerides and diglycerides, petroethrai fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the bacteria of the present invention may be combined with various sweetening or flavouring agents, colouring matter or dyes, with emulsifying and/or suspending agents and with diluents such as water, propylene glycol and glycerin, and combinations thereof.

The forms may also include gelatin capsules; fibre capsules, fibre tablets etc.; or even fibre beverages.

In one aspect, the bacteria according to the present invention may be administered in an aerosol, for example by way of a nasal spray, for instance for administration to the respiratory tract.

### Prebiotics

In one embodiment, the bacterial strains and compositions of the present invention may further be combined or comprise one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route, but it can be also applied to other microbioally colonized sites. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal or skin microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat, unrefined barley and yacon.

Examples of suitable prebiotics include alginate, xanthan, pectin, locust bean gum (LBG), inulin, guar gum, galacto-oligosaccharide (GOS), fructo-oligosaccharide (FOS), polydextrose 10 (i.e. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, (human) milk oligosaccharides and all forms of resistant starches.

The combination of one or more of the bacterial strains according to the present invention and one or more fibres and/or prebiotics according to the present invention exhibits a synergistic effect in certain applications (i.e. an effect which is greater than the additive effect of the bacteria when used separately).

In one embodiment, the bacterial strains or a mixture thereof according to the present invention is used in combination with one or more fibres and/or prebiotic.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, 5 microcrystalline cellulose (MCC), milk oligosaccharide or betaine.

In a further aspect, the invention relates to a composition, food product, a food ingredient, a dietary supplement, a vaccine or a pharmaceutical acceptable composition comprising bacterial strains according to the present invention or a mixture thereof and one or more fibres and/or a prebiotic.

### EXAMPLES

### Example 1—Use of Probiotics to Boost Innate Immunity: Macrophage Activation and Anti-Inflammatory Response

Probiotic strains (DGCC, Danisco Global Culture collection, Niebüll, Germany) were grown to logarithmic growth phase, collected by centrifugation, washed once with PBS (Life Technologies, Paisley, UK), and suspended to cell culture medium. The OD600 was adjusted to correspond to bacteria:host cell ratio of 10:1.

### Monocyte Purification and Differentiation to Macrophages

Monocytes were purified from freshly collected, leukocyte-rich buffy coats obtained from healthy blood donors (Finish Red Cross Blood Transfusion Service, Helsinki, Finland). Human peripheral blood mononuclear cells were isolated by Ficoll-Pague (GE Healthcare) density gradient centrifugation using SepMate tubes (Stemcell technologies, Grenoble, France) followed by purification of monocytes by MACS CD14+ magnetic beads according to manufacturer's instructions (Miltenyi Biotech GmbH, Bergisch Gladbach, Germany). To obtain macrophages purified monocytes were plated on 24 well plates 3×105 cells/well (Falcon, Corning, NY, USA) and cultured 7 d in Macrophage-SFM (Gibco, Life Technologies, Grand Island, NY, USA) with recombinant human GM-CSF (Miltenyi Biotech) 1000 IU/ml and 1% Antibiotic-Antimycotic.

### Stimulation of Immune Cells

Cells from six blood donors were stimulated with probiotic bacteria (bacteria:cell ratio of 10:1). Unstimulated cells were used as a control. Macrophages were stimulated for 24 h, cell culture supernatants were collected and stored at −80° C. until analyzed.

### ELISA Analysis

Cell culture supernatants from macrophage and dendritic cell cultures were analyzed for IFN-g, IL-10, and IL-12p70 by Aushon Human Ciraplex Array (Aushon BioSystems, Inc., Billerica, MA, USA). Results were analyzed with CiraSoft software (Aushon Biosystems).

The cytokine levels were analyzed and normalized across different experiments. The ratio of anti-inflammatory IL-10 and sum of macrophage activating IL-12 and IFN-γ were used as indicator of probiotic effect. The ratio below 0.2 indicates anti-microbial activation of macrophages whereas a ratio higher than 0.2 indicates anti-inflammatory effect (Table 1).

TABLE 1
Macrophage response to probiotics.

| | | | Nor- | |
|---|---|---|---|---|
| | | | malized | |
| | | | IL-10/ | |
| | | | (IFN-γ + | |
| | | | IL-12) | |
| Genus | Species | Strain | ratio | Interpretation |
| Lactiplantibacillus | plantarum | Lp-115 | <0.2 | Macrophage |
| | | | | activation |
| Lactiplantibacillus | plantarum | Lp12418 | Not | Non-activating |
| | | | Detected | |
| Lacticaseibacillus | paracasei | Lpc-37 | <0.2 | Macrophage |
| | | | | activation |
| Lactiplantibacillus | plantarum | Lp12407 | <0.2 | Macrophage |
| | | | | activation |
| Lactobacillus | gasserii | Lg-36 | <0.2 | Macrophage |
| | | | | activation |
| Streptococcus | thermophilus | St-21 | <0.2 | Macrophage |
| | | | | activation |
| Lactobacillus | acidophilus | La-14 | <0.2 | Macrophage |
| | | | | activation |
| Lacticaseibacillus | paracasei | Lc-10 | <0.2 | Macrophage |
| | | | | activation |
| Lacticaseibacillus | rhamnosus | Lr-32 | <0.2 | Macrophage |
| | | | | activation |
| Levilactobacillus | brevis | Lbr-35 | >0.2 | Anti-inflammatory |
| Lactococcus | lactis | Ll-23 | >0.2 | Anti-inflammatory |
| Bifidobacterium | infantis | Bi-26 | >0.2 | Anti-inflammatory |
| Lactobacillus | crispatus | LX1220 | >0.2 | Anti-inflammatory |
| Bifidobacterium | lactis | B420 | >0.2 | Anti-inflammatory |
| Bifidobacterium | longum | Bl-05 | >0.2 | Anti-inflammatory |
| Bifidobacterium | bifidum | Bb-06 | >0.2 | Anti-inflammatory |
| Bifidobacterium | lactis | Bi-07 | >0.2 | Anti-inflammatory |
| Ligilactobacillus | salivarius | Ls-33 | >0.2 | Anti-inflammatory |
| Limosilactibacillus | fermentum | SBS-1 | Not | Non-activating |
| | | | Detected | |
| Limosilactibacillus | reuteri | 1E1 | >0.2 | Anti-inflammatory |
| Lactobacillus | bulgaricus | Lb-87 | >0.2 | Anti-inflammatory |
| Lacticaseibacillus | casei | Lc-11 | Not | Non-activating |
| | | | Detected | |

### Example 2—Use of Probiotics to Modulate Immune Responses by Influencing Dendritic Cell Polarization

Probiotic strains (DGCC, Danisco Global Culture collection, Niebüll, Germany) were grown to logarithmic growth phase, collected by centrifugation, washed once with PBS (Life Technologies, Paisley, UK), and suspended to cell culture medium. The OD600 was adjusted to correspond to bacteria:host cell ratio of 10:1.

### Monocyte Purification and Differentiation to Dendritic Cells

Monocytes were purified from freshly collected, leukocyte-rich buffy coats obtained from healthy blood donors (Finnish Red Cross Blood Transfusion Service, Helsinki, Finland). Human peripheral blood mononuclear cells were isolated by Ficoll-Pague (GE Healthcare) density gradient centrifugation using SepMate tubes (Stemcell technologies, Grenoble, France) followed by purification of monocytes by MACS CD14+ magnetic beads according to manufacturer's instructions (Miltenyi Biotech GmbH, Bergisch Gladbach, Germany). To differentiate monocytes into immature dendritic cells, monocytes were plated on 12 well plates 5×105 cells/well (Falcon, Corning, NY, USA) and cultured for 7 d in RPMI-1640 (Sigma) supplemented with 1% Antibiotic-Antimycotic, 10% fetal bovine serum (FBS) (both from Life Technologies), IL-4 (400 IU/ml), and GM-CSF (1000 IU/ml) (Both from Miltenyi Biotech).

### Stimulation of Immune Cells

Cells from six blood donors were stimulated with probiotic bacteria (bacteria:cell ratio of 10:1). Unstimulated cells were used as a control. DCs were stimulated for 48 h, cell culture supernatants were collected and stored at −80° C. until analyzed.

### ELISA Analysis

Cell culture supernatants from macrophage and dendritic cell cultures were analyzed for IFN-g, IL-6, IL-10, IL-12p70, and IL-23 by Aushon Human Ciraplex Array (Aushon BioSystems, Inc., Billerica, MA, USA). Results were analyzed with CiraSoft software (Aushon Biosystems).

The cytokine levels were analyzed and normalized across different experiments. The ratio of Th1 cytokines (sum of IL-12 and IFN-g) and Th17 cytokines (sum of IL-6 and IL-23) were used as indicator of probiotic effect. As well as production of IL-10 to determine regulatory T cell activation potential (Table 2).

TABLE 2
Dendritic cell cytokine response to probiotics compared to control.

| | | | Probiotic | Probiotic | | |
|---|---|---|---|---|---|---|
| | | | Th1* | Th17* | | |
| | | | cytokine | cytokine | IL-10 | |
| | | | production | production | production | Polarization of |
| Genus | Species | Strain | vs control | vs ctrl | vs control | dendritic cells |
| Lactiplantibacillus | plantarum | Lp-115 | Higher | Higher | Lower | Th1/Th17 |
| Lactiplantibacillus | plantarum | Lp12418 | Higher | Higher | Lower | Th1/Th17 |
| Lacticaseibacillus | paracasei | Lpc-37 | Higher | Higher | Higher | Th1/Th17/Treg |
| Lactiplantibacillus | plantarum | Lp12407 | Higher | Higher | Higher | Th1/Th17/Treg |
| Lactobacillus | gasserii | Lg-36 | Higher | Higher | Higher | Th1/Th17/Treg |
| Streptococcus | thermophilus | St-21 | Higher | Higher | Higher | Th1/Th17/Treg |
| Lactobacillus | acidophilus | La-14 | Higher | Higher | Higher | Th1/Th17/Treg |
| Lacticaseibacillus | paracasei | Lc-10 | Higher | Higher | Lower | Th1/Th17 |
| Lacticaseibacillus | rhamnosus | Lr-32 | Higher | Higher | Similar | Th1/Th17 |
| Levilactobacillus | brevis | Lbr-35 | Higher | Higher | Higher | Th1/Th17/Treg |
| Lactococcus | lactis | Ll-23 | Higher | Higher | Higher | Th1/Th17/Treg |
| Bifidobacterium | infantis | Bi-26 | Lower | Higher | Similar | Th17 |
| Lactobacillus | crispatus | LX1220 | Higher | Higher | Similar | Th1/Th17 |
| Bifidobacterium | lactis | B420 | Higher | Higher | Higher | Th1/Th17/Treg |
| Bifidobacterium | longum | Bl-05 | Higher | Higher | Lower | Th1/Th17 |
| Bifidobacterium | bifidum | Bb-06 | Higher | Higher | Higher | Th1/Th17/Treg |
| Bifidobacterium | lactis | Bi-07 | Higher | Higher | Higher | Th1/Th17 |
| Ligilactobacillus | salivarius | Ls-33 | Higher | Higher | Similar | Th1/Th17 |
| Limosilactibacillus | fermentum | SBS-1 | Lower | Higher | Lower | Th17 |
| Limosilactibacillus | reuteri | 1E1 | Higher | Higher | Higher | Th1/Th17/Treg |
| Lactobacillus | bulgaricus | Lb-87 | Higher | Higher | Higher | Th1/Th17/Treg |
| Lacticaseibacillus | casei | Lc-11 | Higher | Higher | Higher | Th1/Th17/Treg |

*IL-12 + IFN-g
** IL-6 + IL-23

### Example 3—Use of Probiotics to Boost Anti-Viral Immunity: Macrophage Function Boosting and Inflammation Control

Probiotic strains (DGCC, Danisco Global Culture collection, Niebüll, Germany) were grown to logarithmic growth phase, collected by centrifugation, washed once with PBS (Life Technologies, Paisley, UK), and suspended to cell culture medium. The OD600 was adjusted to correspond to bacteria:host cell ratio of 10:1.

### Monocyte Purification and Differentiation to Macrophages

Monocytes were purified from freshly collected, leukocyte-rich buffy coats obtained from healthy blood donors (Finish Red Cross Blood Transfusion Service, Helsinki, Finland). Human peripheral blood mononuclear cells were isolated by Ficoll-Pague (GE Healthcare) density gradient centrifugation using SepMate tubes (Stemcell technologies, Grenoble, France) followed by purification of monocytes by MACS CD14+ magnetic beads according to manufacturer's instructions (Miltenyi Biotech GmbH, Bergisch Gladbach, Germany). To obtain macrophages purified monocytes were plated on 24 well plates 3×105 cells/well (Falcon, Corning, NY, USA) and cultured 7 d in Macrophage-SFM (Gibco, Life Technologies, Grand Island, NY, USA) with recombinant human GM-CSF (Miltenyi Biotech) 1000 IU/ml and 1% Antibiotic-Antimycotic.

### Stimulation of Immune Cells

Cells from six blood donors were stimulated with probiotic bacteria (bacteria:cell ratio of 10:1) in combination with TLR ligand blend, pI:C, 30 μg/ml+R848, 10 μM (both from Sigma-Aldrich, St. Louis, MO, USA) or pI:C+R848 alone. Macrophages were stimulated for 24 h, cell culture supernatants were collected and stored at −80° C. until analyzed.

### ELISA Analysis

Cell culture supernatants from macrophage and dendritic cell cultures were analyzed for IFN-g, IL-10, and IL-12p70, by Aushon Human 6-Plex Ciraplex Array (Aushon BioSystems, Inc., Billerica, MA, USA). In addition, dendritic cell supernatants were analyzed for IL-23 and TGF-b by Aushon Human 1-plex Assays according to manufacturer's instructions. Results were analyzed with CiraSoft software (Aushon Biosystems).

The cytokine levels were analyzed and normalized across different experiments. The ratio of Th1 cytokines (sum of IL-12 and IFN-g) and Th17 cytokines (sum of IL-6 and IL-23) were used as indicator of probiotic effect. IL-10 response was used to determine regulatory T cell activation potential (Table 3).

TABLE 3
Macrophages challenged using pIC + R848 with or without probiotics.

| | | | IL-12 + | | |
|---|---|---|---|---|---|
| | | | IFNg | IL-10 | |
| | | | vs | vs | Inter- |
| Genus | Species | Strain | pICR848 | pICR848 | pretation |
| Lactiplantibacillus | planta- | Lp-115 | Higher | Higher | Anti- |
| | rum | | | | viral |
| | | | | | boosting |
| | | | | | with |
| | | | | | inflam- |
| | | | | | mation |
| | | | | | control |
| Lactiplantibacillus | planta- | Lp12418 | No | No | No |
| | rum | | effect | effect | effect |
| Lacticaseibacillus | para- | Lpc-37 | Higher | Higher | Anti- |
| | casei | | | | viral |
| | | | | | boosting |
| | | | | | with |
| | | | | | inflam- |
| | | | | | mation |
| | | | | | control |
| Lactiplantibacillus | planta- | Lp12407 | Higher | No | Anti- |
| | rum | | | effect | viral |
| | | | | | boosting |
| Lactobacillus | gasserii | Lg-36 | Higher | No | Anti- |
| | | | | effect | viral |
| | | | | | boosting |
| Streptococcus | thermo- | St-21 | Higher | Higher | Anti- |
| | philus | | | | viral |
| | | | | | boosting |
| | | | | | with |
| | | | | | inflam- |
| | | | | | mation |
| | | | | | control |
| Lactobacillus | acido- | La-14 | Higher | Higher | Anti- |
| | philus | | | | viral |
| | | | | | boosting |
| | | | | | with |
| | | | | | inflam- |
| | | | | | mation |
| | | | | | control |
| Lacticaseibacillus | para- | Lc-10 | Higher | No | Anti- |
| | casei | | | effect | viral |
| | | | | | boosting |
| Lacticaseibacillus | rham- | Lr-32 | Higher | No | Anti- |
| | nosus | | | effect | viral |
| | | | | | boosting |
| Levilactobacillus | brevis | Lbr-35 | Lower | Higher | Inhib- |
| | | | | | itory |
| Lactococcus | lactis | Ll-23 | Lower | Higher | Inhib- |
| | | | | | itory |
| Bifidobacterium | infantis | Bi-26 | Lower | Higher | Inhib- |
| | | | | | itory |
| Lactobacillus | cris- | LX1220 | Lower | No | Inhib- |
| | patus | | | effect | itory |
| Bifidobacterium | lactis | B420 | Higher | Higher | Anti- |
| | | | | | viral |
| | | | | | boosting |
| | | | | | with |
| | | | | | inflam- |
| | | | | | mation |
| | | | | | control |
| Bifidobacterium | longum | Bl-05 | Lower | Higher | Inhib- |
| | | | | | itory |
| Bifidobacterium | bifidum | Bb-06 | No | Higher | Inhib- |
| | | | effect | | itory |
| Bifidobacterium | lactis | Bi-07 | Higher | Higher | Anti- |
| | | | | | viral |
| | | | | | boosting |
| | | | | | with |
| | | | | | inflam- |
| | | | | | mation |
| | | | | | control |
| Ligilactobacillus | sali- | Ls-33 | Higher | Higher | Anti- |
| | varius | | | | viral |
| | | | | | boosting |
| | | | | | with |
| | | | | | inflam- |
| | | | | | mation |
| | | | | | control |
| Limosilactibacillus | fer- | SBS-1 | Lower | Higher | Inhib- |
| | mentum | | | | itory |
| Limosilactibacillus | reuteri | 1E1 | Lower | Higher | Inhib- |
| | | | | | itory |
| Lactobacillus | bulga- | Lb-87 | Higher | Higher | Anti- |
| | ricus | | | | viral |
| | | | | | boosting |
| | | | | | with |
| | | | | | inflam- |
| | | | | | mation |
| | | | | | control |
| Lacticaseibacillus | casei | Lc-11 | Higher | Higher | Anti- |
| | | | | | viral |
| | | | | | boosting |
| | | | | | with |
| | | | | | inflam- |
| | | | | | mation |
| | | | | | control |

### Example 4—Use of Probiotics to Boost Anti-Viral Immunity: Dendritic Cell Response

Probiotic strains (DGCC, Danisco Global Culture collection, Niebüll, Germany) were grown to logarithmic growth phase, collected by centrifugation, washed once with PBS (Life Technologies, Paisley, UK), and suspended to cell culture medium. The OD600 was adjusted to correspond to bacteria:host cell ratio of 10:1.

### Monocyte Purification and Differentiation to Dendritic Cells

Monocytes were purified from freshly collected, leukocyte-rich buffy coats obtained from healthy blood donors (Finnish Red Cross Blood Transfusion Service, Helsinki, Finland). Human peripheral blood mononuclear cells were isolated by Ficoll-Pague (GE Healthcare) density gradient centrifugation using SepMate tubes (Stemcell technologies, Grenoble, France) followed by purification of monocytes by MACS CD14+ magnetic beads according to manufacturer's instructions (Miltenyi Biotech GmbH, Bergisch Gladbach, Germany). To differentiate monocytes into immature dendritic cells, monocytes were plated on 12 well plates 5×105 cells/well (Falcon, Corning, NY, USA) and cultured for 7 d in RPMI-1640 (Sigma) supplemented with 1% Antibiotic-Antimycotic, 10% fetal bovine serum (FBS) (both from Life Technologies), IL-4 (400 IU/ml), and GM-CSF (1000 IU/ml) (Both from Miltenyi Biotech).

### Stimulation of Immune Cells

Cells from six blood donors were stimulated with probiotic bacteria (bacteria:cell ratio of 10:1) in combination with TLR ligand blend, pI:C, 30 μg/ml+R848, 10 μM (both from Sigma-Aldrich, St. Louis, MO, USA) or pI:C+R848 alone. DCs were stimulated for 48 h, cell culture supernatants were collected and stored at −80° C. until analyzed.

### ELISA Analysis

Cell culture supernatants from macrophage and dendritic cell cultures were analyzed for IFN-g, IL-6, IL-12p70, and IL-23 by Aushon Human Ciraplex Array (Aushon BioSystems, Inc., Billerica, MA, USA). Results were analyzed with CiraSoft software (Aushon Biosystems).

The cytokine levels were analyzed and normalized across different experiments. The ratio of Th1 cytokines (sum of IL-12 and IFN-g) and Th17 cytokines (sum of IL-6 and IL-23) were used as indicator of probiotic effect.

TABLE 4
Dendritic cells challenged using pIC + R848 with or without probiotics.

| | | | Probiotic | | Probiotic | |
|---|---|---|---|---|---|---|
| | | | Th1* and | | Th1* to | |
| | | | Th17** | | Th17** | |
| | | | cytokine | | cytokine | |
| | | | production | | ratio | |
| | | | compared | | compared | |
| | | | to | Functional | to | Functional |
| Genus | Species | Strain | pI:C + R848 | meaning | pIC + R848 | meaning |
| Lactiplantibacillus | plantarum | Lp-115 | Higher | Immunoboosting | Lower | Th17 |
| | | | | | | polarizing |
| Lactiplantibacillus | plantarum | Lp12418 | Higher | Immunoboosting | Higher | Th1 |
| | | | | | | polarizing |
| Lacticaseibacillus | paracasei | Lpc-37 | Higher | Immunoboosting | Higher | Th1 |
| | | | | | | polarizing |
| Lactiplantibacillus | plantarum | Lp12407 | Higher | Immunoboosting | Higher | Th1 |
| | | | | | | polarizing |
| Lactobacillus | gasserii | Lg-36 | Higher | Immunoboosting | Higher | Th1 |
| | | | | | | polarizing |
| Streptococcus | thermophilus | St-21 | Higher | Immunoboosting | Higher | Th1 |
| | | | | | | polarizing |
| Lactobacillus | acidophilus | La-14 | Higher | Immunoboosting | Higher | Th1 |
| | | | | | | polarizing |
| Lacticaseibacillus | paracasei | Lc-10 | Higher | Immunoboosting | Higher | Th1 |
| | | | | | | polarizing |
| Lacticaseibacillus | rhamnosus | Lr-32 | Higher | Immunoboosting | Higher | Th1 |
| | | | | | | polarizing |
| Levilactobacillus | brevis | Lbr-35 | Higher | Immunoboosting | Similar | Non |
| | | | | | | polarizing |
| Lactococcus | lactis | Ll-23 | Higher | Immunoboosting | Higher | Th1 |
| | | | | | | polarizing |
| Bifidobacterium | infantis | Bi-26 | Higher | Immunoboosting | Higher | Th1 |
| | | | | | | polarizing |
| Lactobacillus | crispatus | LX1220 | Higher | Immunoboosting | Similar | Non |
| | | | | | | polarizing |
| Bifidobacterium | lactis | B420 | Higher | Immunoboosting | Higher | Th1 |
| | | | | | | polarizing |
| Bifidobacterium | longum | Bl-05 | Higher | Immunoboosting | Similar | Non |
| | | | | | | polarizing |
| Bifidobacterium | bifidum | Bb-06 | Higher | Immunoboosting | Higher | Th1 |
| | | | | | | polarizing |
| Bifidobacterium | lactis | Bi-07 | Higher | Immunoboosting | Higher | Th1 |
| | | | | | | polarizing |
| Ligilactobacillus | salivarius | Ls-33 | Higher | Immunoboosting | Lower | Th17 |
| | | | | | | polarizing |
| Limosilactibacillus | fermentum | SBS-1 | Higher | Immunoboosting | Higher | Th1 |
| | | | | | | polarizing |
| Limosilactibacillus | reuteri | 1E1 | Higher | Immunoboosting | Higher | Th1 |
| | | | | | | polarizing |
| Lactobacillus | bulgaricus | Lb-87 | Higher | Immunoboosting | Higher | Th1 |
| | | | | | | polarizing |
| Lacticaseibacillus | casei | Lc-11 | Higher | Immunoboosting | Higher | Th1 |
| | | | | | | polarizing |

*IL-12 + IFN-g
**IL-6 + IL-23

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for boosting an immune system in a subject in need thereof, wherein the method comprises administering to the subject an effective amount of a probiotic composition comprising one or more bacterial strains selected from the species Lactiplantibacillus plantarum, Lacticaseibacillus paracasei, Lacticaseibacillus rhamnosus, Lacticaseibacillus casei, Streptococcus thermophilus, Lactobacillus gasseri, Lactobacillus acidophilus, Lactobacillus crispatus, Lactobacillus bulgaricus, Levilactobacillus brevis, Lactococcus lactis, Bifidobacterium longum subsp. infantis, Bifidobacterium animalis subsp. lactis, Bifidobacterium longum, Bifidobacterium bifidum, Ligilactobacillus salivarius, Limosilactibacillus fermentum and Limosilactibacillus reuteri.

2-5. (canceled)

6. The method according to claim 1, wherein said boosting of the immune system modulates the immune function.

7. The method according to claim 6, wherein the modulation of the immune system boosts anti-pathogen and/or anti-cancer immunity.

8. The method according to claim 6, wherein the modulation of the immune system boosts immunity against extracellular bacteria and fungi.

9. (canceled)

10. The method according to claim 7, wherein said boosting is due to activation of dendritic cells.

11. The method according to claim 10, wherein the dendritic cells activate T-cells.

12. The method according to claim 11, wherein the T-cells are T-helper cell type 1 (Th1) and/or T-helper cell type 17 (Th17) And/or regulatory T-cells (Treg).

13. The method according to claim 1, wherein the boosting of the immune system modulates anti-viral immunity.

14. The method according to claim 13, wherein the modulation of the anti-viral immunity affects anti-viral response.

15. The method according to claim 13, wherein the modulation of the anti-viral immunity inhibits/controls the inflammation during the anti-viral response.

16. The method according to claim 13, wherein the modulation is due to the activation of macrophages.

17. (canceled)

18. The method according to claim 14, wherein the modulation is due to the activation of dendritic cells.

19-20. (canceled)

21. The method according to claim 1, wherein the boosting of the immune system results in antigen specific immune responses.

22. The method according to claim 1, wherein the boosting of the immune system is due to activation of antigen presenting cells.

23. The method according to claim 1, wherein the boosting of the immune system is due to activation of dendritic cells.

24. The method according to claim 1, wherein the boosting of the immune system is due to activation of macrophages.

25. The method according to claim 1, wherein;
the boosting of the immune system is due to activation of antigen presenting cells, dendritic cells and/or macrophages; and
the antigen presenting cells, dendritic cells and/or macrophages induce activation of antigen specific T cells.

26. The method according to claim 25, wherein the antigen specific T cells are T-helper cell type 1 (Th1) and/or T-helper cell type 17 (Th17) and/or regulatory T-cells (Treg).

27-28. (canceled)

29. The method according to claim 1, wherein:
the bacterial strains of the species Lactiplantibacillus plantarum are strains Lp-115, Lp12418 and/or Lp12407;
the bacterial strains of the species Lacticaseibacillus paracasei are strains Lpc-37 and/or Lc-10;
the bacterial strain of the species Lacticaseibacillus rhamnosus is strain Lr-32;
the bacterial strain of the species Lacticaseibacillus casei is strain Lc-11;
the bacterial strain of the species Streptococcus thermophilus is strain St-21;
the bacterial strain of the species Lactobacillus gasseri is strain Lg-36;
the bacterial strains of the species Lactobacillus acidophilus is strain La-14;
the bacterial strain of the species Lactobacillus crispatus is strain Lx1220;
the bacterial strain of the species Lactobacillus bulgaricus is strain Lb-87;
the bacterial strain of the species Levilactobacillus brevis is strain Lbr-35;
the bacterial strain of the species Lactococcus lactis is strain Ll-23;
the bacterial strain of the species Bifidobacterium longum subsp. infantis is strain Bi-26;
the bacterial strains of the species Bifidobacterium animalis subsp. lactis are strain B420 and/or Bi-07;
the bacterial strain of the species Bifidobacterium longum is strain B1-05;
the bacterial strain of the species Bifidobacterium bifidum is strain Bb-06;
the bacterial strain of the species Ligilactobacillus salivarius is strain Ls-33;
the bacterial strain of the species Limosilactibacillus fermentum is strain SBS-1; and
the bacterial strain of the species Limosilactibacillus reuteri is strain 1E1.

30-33. (canceled)

34. The method according to claim 1, wherein said composition is a food product, a food ingredient, a dietary supplement, a vaccine or a pharmaceutical acceptable composition.
